(19) United States
(12) Patent Application Publication
(10) Pub. No.: US 2015/0309015 A1
(43) Pub. Date: Oct. 29, 2015

(54) METHOD FOR DETERMINING COMPATIBILITY BETWEEN A DONOR AND A RECIPIENT BY MEANS OF THE FLOW CYTOMETRIC DETECTION OF ALLOREACTIVE T CELLS

(71) Applicant: Lophius Biosciences GmbH, Regensberg (DE)

(72) Inventors: Ferdinand Hermann BAHLMANN, Hannover (DE); Danilo FLISER, Heidelberg (DE); Martina SESTER, Homburg (DE); Urban SESTER, Homberg (DE)

(21) Appl. No.: 14/648,319

(22) PCT Filed: Dec. 2, 2013
(86) PCT No.: PCT/EP2013/075253

§ 371 Date: May 29, 2015

(30) Foreign Application Priority Data
Nov. 30, 2012 (EP) 12195029.9

(51) Int. Cl. G01N 33/50

(52) U.S. Cl. CPC G01N 33/5091

(57) ABSTRACT

Method for determination of compatibility between a donor and a recipient by flow cytometric detection of allo-reactive T cells comprising the following steps:
a) addition of a marker to a obtained blood sample from a donor/recipient pair to be analyzed, called labelled sample in the following, wherein the marker is suitable for discrimination of a sample from the recipient; the other blood sample obtained from donor/recipient pair to be analyzed is called in the following non-labelled sample,
b) at least one-time dilution of both samples with a first medium, separation of cellular parts of the samples from the liquid phase,
c) respective separated reuptake of the cellular parts, in particular in the same volume, of the blood sample residue of the labelled sample, of the blood sample residue of the non-labelled sample in a second medium
d) mixing of a part from the non-labelled sample and a part of the labelled sample, in particularly in a ratio of about 1:1 under preservation of a mixture,
e) stimulation of antigen presenting cells (APC) in a mixture of labelled and non-labelled sample according to step d) of claim 1 by addition of anti-CD28− antibodies or anti-CD28− antibodies and anti-CD49d-antibodies followed by
f) a first incubation of the mixture for the period of 1.5 h to 2.5 h, preferably 2 h, at a temperature of 35-39° C., preferably 37° C., optional under protective atmosphere, CO₂ atmosphere,
g) addition of a secretion inhibitor,
h) mixing of the sample of step d) of claim 1,
i) followed by a second incubation for a period of at least 2.5 h at a temperature of 35-39° C., preferably 37° C., optional under protective atmosphere, CO₂ atmosphere,
j) if necessary lysing of erythrocytes,
k) flow cytometric detection of lymphocytes by marking and flow cytometric detection of CD4 or CD8 lymphocytes e.g. by labelled anti-CD4 and/or anti-CD8 antibodies
l) flow cytometric detection of lymphocytes activated by a intracellular cytokine, activated lymphocytes and lymphocytes expressing an activation marker,
m) wherein a present allo-reactivity was detected between the individual, from which the labelled sample and that from which the non-labelled sample was obtained, if in the flow cytometric detection the following results occur:
   a. marking pos/CD4 pos/CD69 pos/INFgamma pos,
   b. marking neg/CD4 pos/CD69 pos/INFgamma pos,
   c. marking pos/CD8 pos/CD69 pos/INFgamma pos,
   d. marking pos/CD8 pos/CD69 pos/INFgamma

The object of the invention is a method for determination of compatibility between a donor and a recipient by flow cytometric detection of allo-reactive T cells.

The cell-mediated immune reaction against donations of tissues or organs plays an important role both in the acute and chronic transplant rejection. It is of great importance to estimate the risk, if a rejection of the donation occurs in the recipient.

Urban Sester et al. describe in Transplantation, Vol. 78 (4), 607-614, (2004) a method for identification of allo-reactive T cells for use in the clinical practice. The disadvantage is the relatively complex methodology using peripheral blood mononuclear cells.

It is thus desirable to provide a simpler test which allows a fast and secure compatibility risk estimate, particularly from whole blood.

This is possible according to the present invention by the method for determination of compatibility between a donor and a recipient by flow cytometric detection of allo-reactive T cells. The method according to the present invention comprises the following steps:
a) addition of a marker to a obtained blood sample from a donor/recipient pair to be analyzed, called labelled sample in the following, wherein the marker is suitable for discrimination of a sample from the recipient; the other blood sample obtained from donor/recipient pair to be analyzed is called in the following non-labelled sample,
b) at least one-time dilution of both samples with a first medium, separation of cellular parts of the samples of the liquid phase,
c) respective separated reuptake of the cellular parts, in particular in the same volume, of the blood sample residue of the labelled sample, of the blood sample residue of the non-labelled sample in a second medium
d) mixing of a part from the non-labelled sample and a part form the labelled sample, in particularly in a ratio of about 1:1 under preservation of a mixture,
e) stimulation of antigen presenting cells (APC) in a mixture of labelled and non-labelled sample according to step d) of claim 1 by addition of anti-CD28-antibodies or anti-CD28-antibodies and anti-CD49d-antibodies followed by
f) a first incubation of the mixture for the period of 1.5 h to 2.5 h, preferably 2 h, at a temperature of 35-39° C., preferably 37° C., optional under protective atmosphere, CO₂ atmosphere,
g) addition of a secretion inhibitor,
h) mixing of the sample of step d) of claim 1,
i) followed by a second incubation for a period of at least 2.5 h at a temperature of 35-39° C., preferably 37° C., optional under protective atmosphere, CO₂ atmosphere,
j) if necessary lysing of erythrocytes,
k) flow cytometric detection of lymphocytes by marking and flow cytometric detection of CD4 or CD8 lymphocytes e.g. by labelled anti-CD4 and/or anti-CD8 antibodies
l) flow cytometric detection of lymphocytes activated by a intracellular cytokine and lymphocytes expressing an activation marker,
m) wherein a present allo-reactivity was detected between the individual, from which the labelled sample and that from which the non-labelled sample was obtained, if in the flow cytometric detection the following results occur:
   a. marking pos/CD4 pos/CD69 pos/INFgamma pos,
   b. marking neg/CD4 pos/CD69 pos/INFgamma pos,
   c. marking pos/CD8 pos/CD69 pos/INFgamma pos,
   d. marking pos/CD8 pos/CD69 pos/INFgamma pos.

In one embodiment of the method according to the present invention the sample or both samples may be derived from whole blood.

In a further embodiment of the method according to the present invention the marking is a labelled CD45 antibody, CFDA-SE or CFSE.

In particularly the first medium used in step b) of the method according to the present invention may be independent of the second medium mentioned in step c) of claim 1. The first medium and the second medium may be any cell culture media, in particular such, which are selected from the group consisting of phosphate buffered saline solution, RPMI.

In the method according to the present invention the separation of cellular components of the samples from the liquid phase may be performed using centrifugation.

Typically the secretion inhibition is brefeldin A (BFA) or monesin.

In even another embodiment of the method according to the present invention the cell membrane permeator added prior to the flow-cytometric detection is a saponin, TweenR.

As an activation marker CD69 may be used.

Upon the result of step m) case a. allo-reactive CD4 T-cells of the labelled partners of the donor/recipient pair are shown up against the non-labelled partner from the donor/recipient pair.

Upon the result of step m) case b. allo-reactive CD4 T-cells of the non-labelled partners of the donor/recipient pair are shown up against the labelled partner from the donor/recipient pair.

Upon the result of step m) case c. allo-reactive CD8 T-cells of the labelled partners of the donor/recipient pair are shown up against the non-labelled partner from the donor/recipient pair.

Upon the result of step m) case d. allo-reactive CD8 T-cells of the non-labelled partners of the donor/recipient pair are shown up against the labelled partner from the donor/recipient pair.

In these cases there exists an increased risk of rejection.

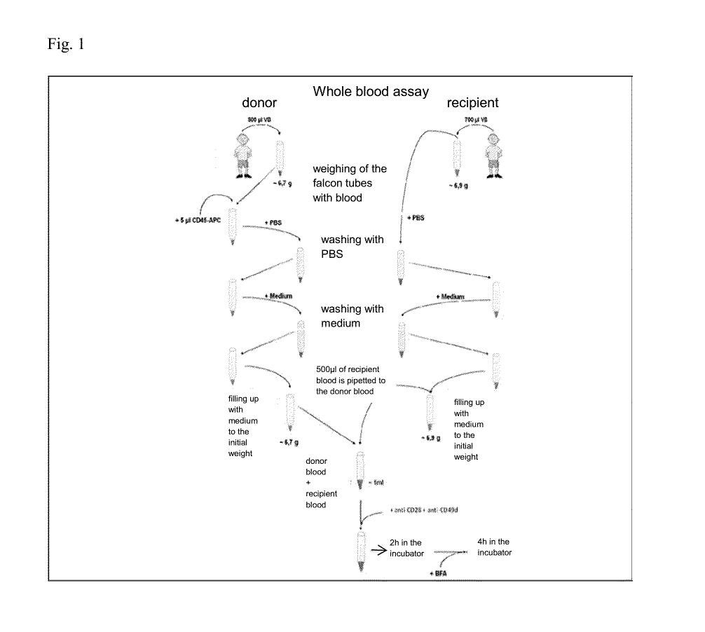

FIG. 1: Sketchy presentation of the pre-staining and stimulation in the whole blood assay. Based on 500 μl whole blood (VB) of the donor and 700 μl whole blood of the recipient the initial weight of the Falcon-tube is determined. Subsequently 5 μl CD45-APC antibody is added to the donor blood and incubated for 30 min in dark (at room temperature). In the next step both the recipient and the donor blood sample is washed with 15 ml PBS (centrifugation at 353 g for 10 min). After removal of the supernatant it is washed with 15 ml medium (RPMI; 1% Gln; 1% P/S; 0.5% HSA). After centrifugation (353 g; 10 min) the supernatant is removed and the Falcon-tubes filled up with medium to their initial weight. Then 500 μl of the recipient blood (undyed blood sample) is pipetted to the donor blood (˜1 ml). For stimulation a mixture of anti-CD28 and anti-CD49d is pipetted in addition and incubated for 2 h in the incubator. Subsequent BFA is added to the sample and is incubated for 4 h in the incubator (curved arrows added).

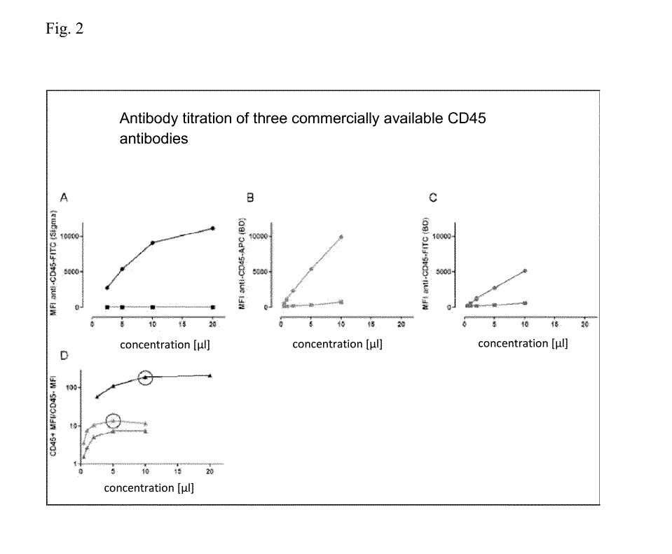

FIG. 2: It is applied in each case the medium fluorescence intensity (MFI) of the CD45 dyed (round symbols) and CD45 undyed cell population (square symbols) of each AK A) CD45-FITC from Sigma (blue), B) CD45-APC from BD (brown) and C) CD45-FITC from BD (green) in dependence of the manufacturer's concentrations and respective dilutions (½, ¼ and ⅛ of each concentration). The MFI of the dyed cell population remains on a zero level (A) and increases slightly (B and C), respectively. In figure D) the ratio of the MFI of CD45-positive cell population to the MFI of CD45-negative cell population is applied. The anti-CD45-FITC from Sigma (blue) shows the highest MFI ratio. The anti-CD45-APC from BD (brown) shows an even better ratio of the MFIs, compared to the CD45-FITC from BD (green). The red circles represent those concentrations, for which it was decided in the further experiment.

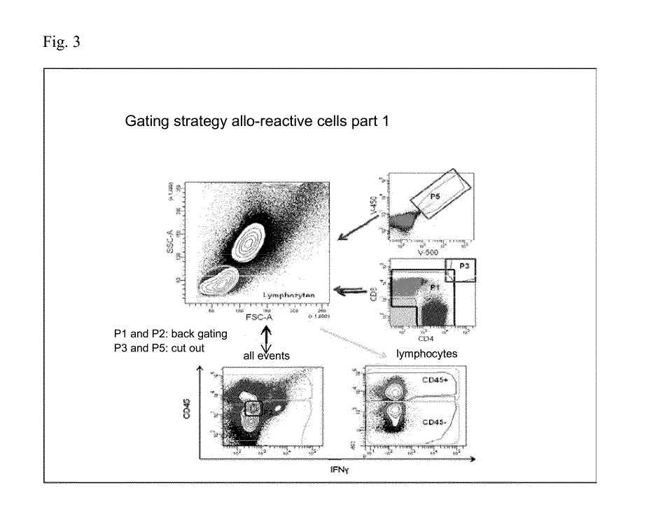

FIG. 3: Analysis of the FACS measurement part 1. The analysis strategy is applied in whole blood samples and PBMC samples in general upon the same algorithm. At first unspecific events represented in the gate P5 are removed by the representation of two non-occupied channels (V-450 and V-500) against each other (dot blot above on the right). In the Dotblot below CD8 is applied against CD4. Here the cells lying in gate P3 are cut out. The cells lying in the gate P1 are “gated back”, i.e. on the left side (FSC against SSC) in the dot blot representation only cells are presented which are visible in the gate P1 (middle dot blot, right side). A second “back gating” on the lymphocytes takes place via the gate P2 (density blot, below on the left side), in which it was gated on a population between CD45 positive and CD45 negative cells (blue gate). Since these cells due to the localization in the density blot (above on the left, presented in blue) are no lymphocytes, it was envisaged in the gating to not include them in the evaluation. In the density blot (above on the left) on which both back gating strategies were applied, the forward scatter (FSC) is represented against the sideward scatter (FSC). Thereby it is possible to gate on the lymphocyte population (frame). The lymphocytes are further classified in CD45 positive (frame, above) and CD45 negative (frame, below) cells by a density blot representation (right side below).

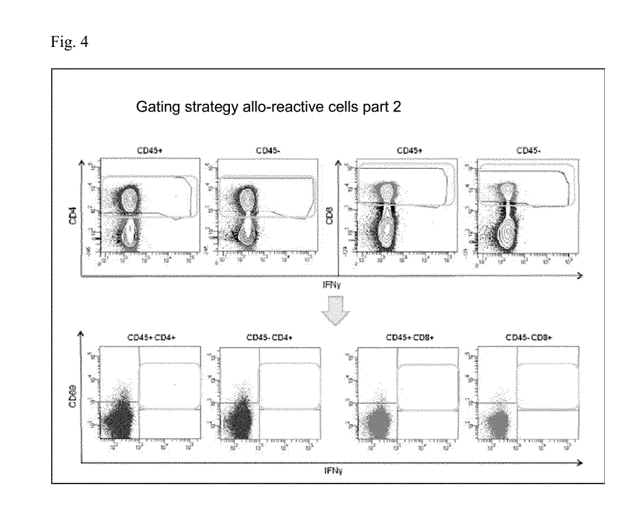

FIG. 4: Analysis of the FACS measurement part 2. In the next step the CD45 positive and CD45 negative lymphocytes from part 1 are further classified in CD4 positive (the two left squares) and CD8 positive (the two right squares) cells, which are applied against IFNγ (upper four squares). In the four dot blots below only the positive T cells of the above density blots are presented, thus only the cells which are in the yellow frame. Here the both left dot plots represent the CD4 positive T cells again; the both on the right represent CD8 positive T cells. These cells are in the following classified due to their expression of the activation marker CD69 and the cytokine IFNγ in four subpopulations, wherein each in the upper right quadrant (frame) T cells defined as allo-reactive are lying.

CD45+CD4+: CD4 T cells of the donor. The events in the right upper quadrant correspond to the allo-reactive CD4 T cells of the donor, which react against the cells of the recipient.

CD45−CD4+: CD4 T cells of the recipient. The events in the right upper quadrant correspond to the allo-reactive CD4 T cells of the recipient, which react against the cells of the donor.

CD45+CD8+: CD8 T cells of the donor. The events in the right upper quadrant correspond to the allo-reactive CD8 T cells of the donor, which react against the cells of the recipient.

CD45−CD8+: CD8 T cells of the recipient. The events in the right upper quadrant correspond to the allo-reactive CD8 T cells of the recipient, which react against the cells of the donor.

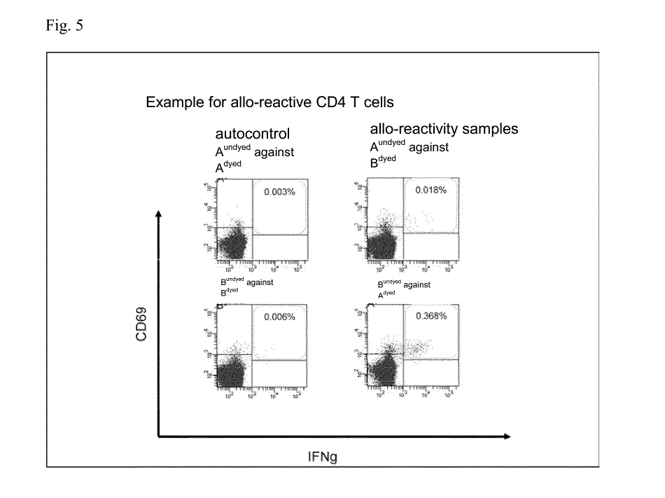

FIG. 5: Represented are each the IFNg/CD69-dot plots of undyed CD4 T cells of four distinct preparations conducted in parallel (each two allo-reactivity samples and the corresponding autocontrols). The allo-reactivity samples are shown in the right column, the corresponding autocontrols are shown in the left column. Thereby 0.015% of the CD4 T cells of the test person A show an allo-reactivity against the test person B (autocontrol deducted). In contrast 0.362% of the CD4 T cells of the test person B show an allo-reactivity against the test person A (autocontrol deducted).

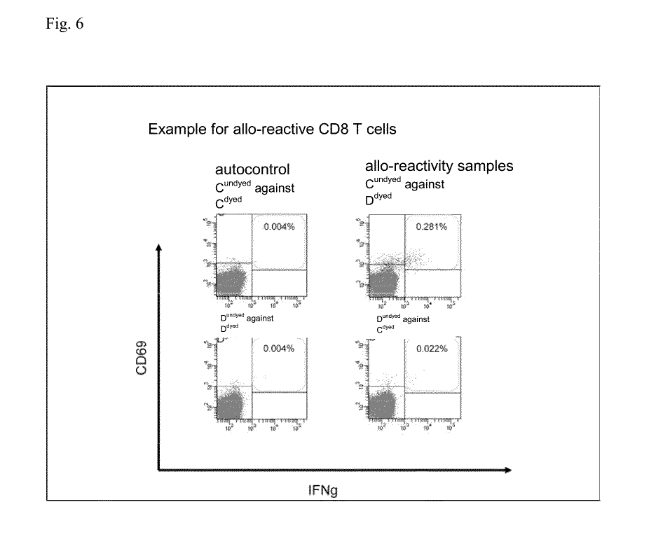

FIG. 6: Represented are each the γ-IFN/CD69-dot plots of undyed CD8 T cells of four distinct preparations conducted in parallel (each two allo-reactivity samples and the corresponding autocontrols). The allo-reactivity samples are shown in the right column, the corresponding autocontrols are shown in the left column. Thereby 0.277% of the CD8 T cells of the test person C show an allo-reactivity against the test person D (autocontrol deducted). In contrast 0.018% of the CD8 T cells of the test person D show an allo-reactivity against the test person C (autocontrol deducted).

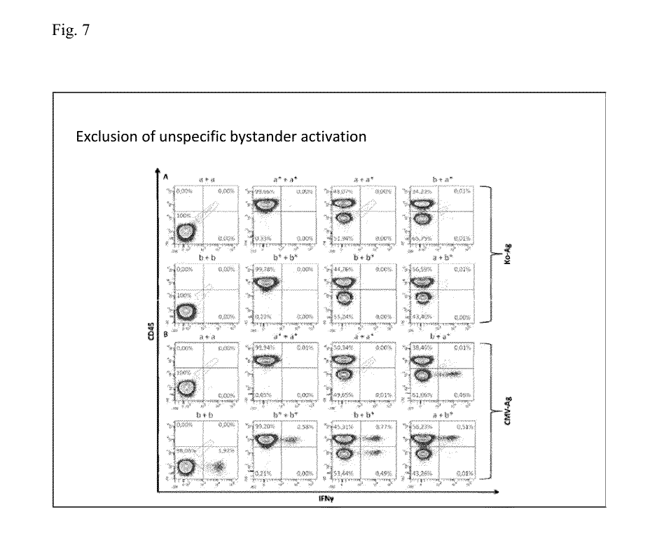

FIG. 7: Represented are the CD4 T cells of the cytomegalovirus (CMV) seronegative test person a and the CMV-seropositive test person b. The dot plots show the γ-IFN expression of the CD45− partially dyed (*) and undyed autologous stimulations and the allo-measurement of test person a and b (from left to right) in representation (A) after stimulation with a control antigen and in representation (B) after simulation with CMV-Ag. (A) After unspecific stimulation no γ-IFN expression occurs. (B) After stimulation with CMV-Ag only in CMV-seropositive test person b a γ-IFN expression can be detected. Unspecific cellular debris (grey gate) have been not involved in the analysis. The cell frequencies are given in the single quadrants in %.

Based on a pre-experiment also a combination of blood samples with different blood groups seems not to lead to a loss of allo-reactivity or to falsified T cell frequencies, since both in combinations with same AB0 blood groups and in test persons with different blood groups allo-reactive T cells have been detected. This indicates that isoagglutinins in the whole blood assay do not play a role and also are removed by the washing steps, respectively.

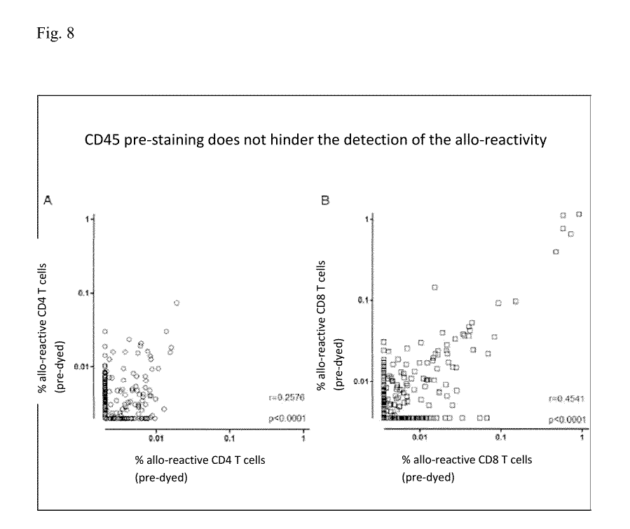

FIG. 8: Represented are 590 pair combinations of 47 test persons, wherein a test person exists as “recipient” undyed and the same test person one time in the same pair combination as before exists as “donor” pre-dyed for CD4 (A) and CD8 (B) T cells. In both representations a significant correlation between the onset of the one test person as undyed and the other as pre-dyed is recognized (CD4: Spearman test, r=0.2576, p<0.0001; CD8: Spearman test, r=0.4541, p<0.0001).

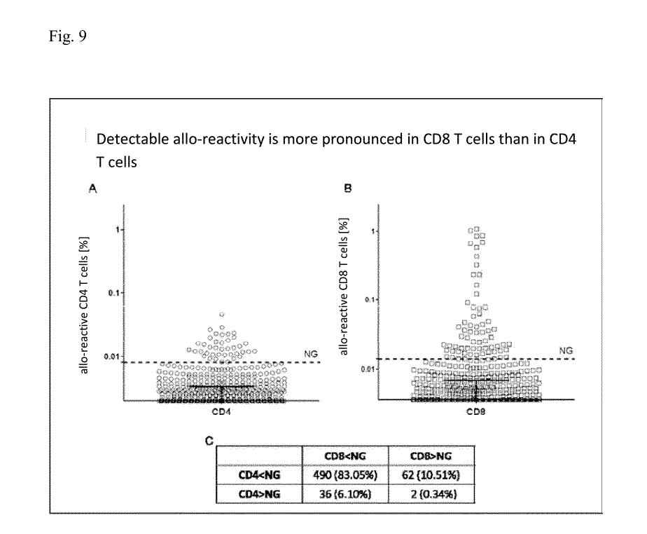

FIG. 9: 590 independent pair combinations of 47 test persons were checked for the presence of all-reactive CD4 T cells (A) and allo-reactive CD8 T cells (B) in whole blood assay. Thereby the calculated detection limit (NG, dashed line) is 0.0081% for allo-reactive CD4 T cells and 0.0145% for allo-reactive CD8 T cells. (C) Ration of the different pair combinations of the allo measurements over and below the detection limit (Fishers exact test, p=0.4149). The data are presented as median±IR.

For detection of allo-reactive T cells in whole blood heparinized whole blood was required from each two test persons (“recipient and donor”). Similar to the use of isolated PBMC the donor blood sample was pre-dyed with CD45 antibody, to differentiate the cells of the both individuals in the flow cytometer after addition of the donor and recipient blood.

The pretreatment of the cells of the recipient and donor was each performed in a 15 ml falcon-tube. Similar to the use of PBMC, the recipient sample remained undyed and was added to the donor sample pre-dyed with anti-CD45 (FIG. 1). Since a pipetting loss occurred during the washing procedures of the pretreatments, in the recipient samples 700 μl whole blood was used, from which 500 μl were added to 500 μl whole blood of the donor.

The falcon-tubes with whole blood were weight initially and the initial weight (6.9 g in recipient samples and 6.7 g in donor samples) were noted. For the staining of the donor samples 5 μl of CD45 antibody was pipetted directly in each tube, mixed and incubated for 30 min in dark. Subsequently, both the recipient and donor samples were washed with each 14 ml PBS (centrifugation: 353 g; 10 min). The supernatant was removed and the sample was washed with 14 ml medium (RPMI+0.5% HAS+1% P/S) (centrifugation: 353 g; 10 min).

The single washing steps were necessary to remove the isoagglutinitns (blood group antibodies), which might effect a disorder of the allo-reactivity in the measurement. After careful removal of the supernatant the 15 ml falcon-tubes were again weight and filled with medium to reach their initial weight. Afterwards 500 μl of the recipient sample of a preparation were transferred to the corresponding donor sample of the same preparation. The complete amount of cell suspension and whole blood, respectively in a preparation was now 1 ml (FIG. 1). To each stimulation preparation, a mixture of costimulatory antibodies (1 μg/ml anti-CD28 and 1 μg/ml anti-CD49d) is added. The stimulation preparations were mixed and subsequently incubated for 2 h in the incubator (37° C., 6% CO₂, with loose screwed cover). Following up the fixation of the whole blood preparations were performed.

In selected experiments, which contributed to the method characterization and establishment a stimulation with control (Ko), CMV-antigens each 32 μg/ml or with Staphylococcus aureus Enterotoxin B (SEB, 2.5 μg/ml) additionally to the allo-reaction was performed. They were added after the addition of costimulatory antibodies and afterwards a two hour incubation in the incubator was performed as described above, with subsequent addition of BFA and a further incubation for 4 h in the incubator. Upon stimulation with CMV a test person shows a respective immune reaction, if it had contact to CMV before and as a result of a specific T cell immune response has been developed. The stimulation with control antigens served as negative control and the one with SEB as positive control. SEB is a super antigen, which results at low concentration already in a strong stimulation. Thereby the T cells were stimulated independent of their antigen specificity. Due to the positive control it was checked if the stimulation conditions are correct and if the T cells of a test person may be stimulated in principle, respectively.

For fixation of the whole blood preparations, 2 mM EDTA was added first which loses the cell-cell-contact and the tubes were mixed on a shaker for each 10 s. After incubation for 15 min at RT in the dark 9 ml Lysing solution per ml whole blood was added for the lysis of the erythrocytes and for fixation of the leukocytes and after further incubation for 10 min (in dark at RT) and subsequent centrifugation (10 min at 353 g) the supernatant was sucked off and the cells were resuspended in 2 ml FACS buffer. A second centrifugation step (10 min at 353 g) followed and a subsequent removal of the supernatant. Then the pellet was given in 400 μl/sample FACS buffer. Following up the cells were either dyed directly or stored in the freezer over night at 4° C.

For the flow cytometric staining (FACS staining) each 200 μl of the cell suspension to be analyzed was transferred in FACS tubes and was incubated in 2 ml saponin buffer for 10 min. The incubation was performed at RT in the dark, since the samples were already pre-dyed with a fluorochrome-coupled surface antibody, which would be bleached out in the light. Due to the saponin the cells were permeabilized so that the added AK in the following might get in the cells. After incubation the tubes were centrifuged for 10 min at 353 g and subsequently the supernatant was removed. 50 μl AK mixture per sample were pipetted (see table 1), mixed and incubated for 30 to 45 min. Then the cells were washed with 3 ml FACS buffer, to remove free, unbound fluorochrome-coupled AK. After the centrifugation of the samples for 7 min at 353 g the supernatant was sucked off and finally, the cells were given in 400 μl 1% PFA, mixed and analyzed in the flow cytometer.

TABLE 1
Composition of the antibody mixtures for different stainings

| Staining 1 | | | Staining 2 | | |
|---|---|---|---|---|---|
| | | Amount | | | Amount |
| antibody | conjugate | [μl] | Antibody | conjugate | [μl] |
| CD8 | APC | 0.5 | CD8 | PerCP | 4 |
| CD4 | PE-Cy7 | 0.5 | CD4 | PE-Cy7 | 0.5 |
| CD69 | PerCP | 2 | CD69 | PE | 2 |
| IFNγ | PE | 0.3 | IFNγ | FITC | 0.5 |
| SAP 5% | | 1 | SAP 5% | | 1 |
| FACS | | 47.7 | FACS | | 42 |
| buffer | | | buffer | | |
| Sum | | 50 | Sum | | 50 |

The flow cytometry (FACS, Fluorescence activated cell sorting) is a laser-based method for determination of specific light scattering properties and fluorescence properties of cells, which were labelled with an appropriate antibody before.

Via a capillary the cell suspension was placed in a measuring cuvette by overpressure. Due to hydrodynamic focusing the cells were strongly accelerated by a surrounding carrier liquid so that single cells pass sequentially the laser beam at the analysis point, where they are collected, analyzed and classified. When a cell interacts with a laser beam, a diffraction and scattering of the light beam occurred typical for a cell population, which is detected by photo detectors. The so-called forward scatter light (FSC=forward scatter) provides information about the size of the cells and is caused by the diffraction of the light. The side scatter light (SSC=side scatter) provides information about granularity of the cells and is caused by the refraction of the light. The size properties and granularity properties of any cell may be reflected in a two dimensional diagram and serve for the identification of the cell subpopulations.

Single cells of a population or subpopulation may be identified and characterized in more detail by antibodies additionally coupled with a fluorochrome. This may be done by staining of cell type specific surface molecules (membrane receptors, for example CD3, CD4 or CD8) or after permeabilisation of the cell membrane by detection of intracellularly located antigens. The fluorochromes were stimulated by the laser and emit the light in form of photons of longer wave lengths, which may be measured by an optical system. Since different fluorochromes have different characteristic emission wave length despite of same activation wave lengths, it is possible to discriminate several distinct coupled AK with a laser and thereby analyze different cell properties.

The fluorochrome FITC (Fluorescein isothiocyanate) was applied among others, which emits fluorescences of maximal 520 nm stimulated by an argon laser (blue) in the wave length region of 488 nm. Phycoerythrin (PE) with a maximal emission of 576 nm was also applied. PerCP (peridinin chlorophyll protein complex) is visible at 678 nm and PE-Cy7 (phycoerythrin-cyanine 7) at 785 nm. In contrast, allophycocyanine (APC) is stimulated by a helium neon laser (red) at 635 nm wave length. The emission maximum of this dye is at 660 nm.

For the measurement and analysis and for the computer-supported data processing a flow cytometer (FACS Canto TI) and the software program FACS Diva Software version 6.1.3 was used. The classification of the single cells was performed by a “dot plot” and “density plot” representation of scatter light signals in forward scatter (FSC) and sider scatter (SSC) light. Thereby both in whole blood and in the PBMCs CD4 and CD8 T cells were collected flow cytometric per sample and analyzed by a defined gating strategy. To ensure a sufficient cell number, it was aimed to collect preferably 100.000 CD8 T cells per measurement.

As a result, the cells were first graphically represented by their granularity (SSC) and their size (FSC) and the lymphocyte region was localized (“gated”). One point corresponds in the “dot plot” representation to a single cell and the density gradient in the “density plot” representation to the frequency of cells of a specific property. In the following the lymphocytes were classified in CD45 pre-dyed (“donor” and CD45+ respectively) and undyed (“recipient” and CD45− respectively) cells (FIG. 3). These were further classifies in CD8 and CD4 positive T cells (FIG. 4). For further collection of functional properties the four cell populations (CD4+CD45+; CD4+CD45−; CD8+CD45+; CD8+CD45−) were classified in each four subpopulations using the expression of the activation marker CD69 and the cytokine IFNγ. The percentual part of the fluorescence signals in the single quadrants could be determined by the division in the quadrants. Allo-reactive T cells were defined by coexpression of CD69 and IFNγ and thus occurred in the upper right quadrant (FIG. 5 and FIG. 6).

The statistical analysis in this work was performed using the GraphPad prisms program. The not parametric Mann-Whitney test was used if continued variables of two groups were compared. For the respective analyses of more than two groups the Kruskal-Wallis test was used and if a significance occurred the single collectives were compared using the Dunn's Multiple Comparison test. The dependency for two or more categorical variables were determined using the Fisher's exact test and Chi Square test, respectively. For correlation analyses the Spearman test was used. All p values below 0.05 were considered as significant.

1. A method for determining compatibility between a donor and a recipient by flow cytometric detection of allo-reactive T cells comprising the following steps:
providing a first blood sample obtained from a donor/recipient pair to be analyzed and adding a marker to said first blood sample to create a labelled sample, wherein the marker is suitable for discrimination of a sample from the recipient and providing second blood sample obtained from said donor/recipient pair to create a non-labelled sample,
diluting both samples with a first medium, and separating cellular parts of both samples from the liquid phase,
c) performing respective separated reuptake of the cellular parts of both samples in a second medium,
d) mixing a portion of the non-labelled sample and a portion of the labelled sample, to create a mixture,
e) stimulating antigen presenting cells (APC) in the mixture step d) by adding anti-CD28-antibodies or anti-CD28-antibodies and anti-CD49d-antibodies,
f) incubating the mixture of step e) for 1.5 to 2.5 hrs, at a temperature of 35-39° C.,
g) adding a secretion inhibitor to the mixture of step f),
h) mixing of the sample,
i) incubating the mixture of step h) for a period of at least 2.5 hrs at a temperature of 35-39°,
j)
detecting, using flow cytometry, CD4 or CD8 lymphocytes, and
l) detecting, using flow cytometry, lymphocytes activated by a intracellular cytokine, and lymphocytes expressing an activation marker,
wherein allo-reactivity is detected between the individual from which the labelled sample was obtained and the individual from which the non-labelled sample was obtained, if in the flow cytometric detection the following results occur:
1) marking pos/CD4 pos/CD69 pos/IFNγ pos,
2) marking neg/CD4 pos/CD69 pos/IFNγ pos,
3) marking pos/CD8 pos/CD69 pos/IFNγ pos, or
4) marking pos/CD8 pos/CD69 pos/IFNγ pos.

2. The method according to claim 1, wherein the marker of step a) is a labelled CD45 antibody, CFD-SE or SFSE.

3. The method according to claim 1, wherein the first medium of step b) of claim 1 is independent of the second medium of step c) of claim 1 and the first medium and the second medium are selected from the group consisting of phosphate buffered saline solution, RPMI.

4. The method according to claim 1, wherein the separation of cellular components of the samples from the liquid phase is performed using centrifugation.

5. The method according to claim 1, wherein the secretion inhibitor is brefeldin A (BFA) or monesin.

6. The method according to claim 1, wherein the cell membrane permeator is a saponin.

7. The method according to claim 1, wherein the cytokine is IFNγ.

8. The method according to claim 1, wherein the activation marker is CD69.

9. The method according to claim 1, wherein 1) shows allo-reactive CD4 T-cells of the labelled partners of the donor/recipient pair against the non-labelled partner from the donor/recipient pair.

10. The method according to claim 1, wherein 2) shows allo-reactive CD4 T-cells of the non-labelled partner of the donor/recipient against the labelled partner from the donor/recipient pair.

11. The method according to claim 1, wherein 3) shows allo-reactive CD8 T-cells of the labelled partner of the donor/recipient pair against the non-labelled partner from the donor/recipient pair.

12. The method according to claim 1, wherein 4) shows allo-reactive CD8 T-cells of the non-labelled partner of the donor/recipient pair against the labelled partner from the donor/recipient pair.

13. The method according to claim 1, wherein the donor blood samples and recipient blood samples are derived from whole blood.

14. The method according to claim 1, further comprising lysing erythrocytes after step i), and before step j).

15. The method according to claim 1, wherein step b) is performed more than once.

16. The method according to claim 1, wherein step c) uses a mixture of the respective cellular parts in the same volume.

17. The method according to claim 1, wherein in step d), the respective portions are mixed in a ratio of about 1:1.

18. The method according to claim 1, wherein step f) comprises incubating for 2 hrs, at 37° C., and/or under a protective atmosphere, CO₂ atmosphere.

19. The method according to claim 1, wherein step i) comprises incubating at 37° C., and/or under a protective atmosphere, CO₂ atmosphere.

20. The method according to claim 1, wherein marking in step j) comprises labelling with anti-CD4 and/or anti-CD8 antibodies.
